(19) United States
(12) Patent Application Publication
(10) Pub. No.: US 2006/0127909 A1
(43) Pub. Date: Jun. 15, 2006

(54) Polynucleotides targeted against hter and use thereof

(76) Inventors: Bernd Schwenzer, Bannewitz (DE); Uta Schmidt, Dresden (DE); Manfred Wirth, Dresden (DE); Kai Kramer, Dresden (DE); Susanne Fussel, Dresden (DE); Axel Meye, Dresden (DE)

Correspondence Address: NORRIS, MCLAUGHLIN & MARCUS, P.A., 875 THIRD AVE, 18TH FLOOR, NEW YORK, NY, 10022, US

(21) Appl. No.: 10/537,449

(22) PCT Filed: Dec. 8, 2003
(86) PCT No.: PCT/DE03/04114

§ 371 Date: Jan. 9, 2006

(30) Foreign Application Priority Data
Dec. 6, 2002 (DE) 102 58 117.7
Feb. 7, 2003 (DE) 103 06 084.7

(51) Int. Cl. A61K 48/00; C12Q 1/68; C07H 21/04

(52) U.S. Cl. 435/6; 514/44; 536/23.2

(57) ABSTRACT

The present invention relates to polynucleotides directed towards a gene of a catalytic subunit of human telomerase, as well as to the use of said polynucleotides for the diagnosis, prophylaxis, reduction and follow-up of diseases associated with cell growth, differentiation and/or division, such as tumor diseases.

The present invention relates to polynucleotides directed towards a gene of a catalytic subunit of human telomerase (hTERT), as well as to the use of said polynucleotides for the diagnosis, prophylaxis, treatment and follow-up of diseases associated with cell growth, differentiation and/or division, such as tumor diseases.

Replication of the ends of eukaryotic chromosomes is known to require specialized cell components. Replication of a linear DNA strand normally proceeds in 5′-3′ direction. Due to the removal of the RNA primers complementary to the outer 3′ end of the chromosomal DNA, said RNA primers being essential in initiating replication, the 5′ ends of newly synthesized DNA strands remain incomplete after each replication cycle. This results in a continuous reduction in length of the daughter strands in each replication cycle (end replication problem) [Levy et al.]. Inter alia, said reduction in length at the chromosome ends—being referred to as telomeres—is responsible for the control of the proliferative capability and thus for ageing of cells [Harley]. The structure of said telomeres has been investigated in numerous living systems.

In a variety of organisms the telomerase ribonucleo-enzyme assumes the function of extending and stabilizing the telomeres of proliferating cells, thereby levelling the end replication problem [Greider et al.]. The above reverse transcriptase consists of two essential subunits: an RNA component (hTR) and a catalytic subunit (hTERT) [Beattie et al.]. In accordance with the relationship between telomeres and telomerase and the proliferative capability of cells, telomerase activity has been detected in immortalized cell lines, as well as in >85% of tumors that have been investigated [Kim et al.]. The latter correlates with the expression of the hTERT component, as has been demonstrated in bladder carcinoma [Ito et al.]. Furthermore, the hTERT expression levels in bladder carcinoma are known to be associated with the clinical course of the tumor disease [de Kok et al.]. Therefore, human telomerase is an ideal target in the diagnosis and treatment of human diseases associated with cellular proliferation, such as cancer. Inter alia, methods for the diagnosis and treatment of cancer and other diseases associated with telomerase have been disclosed in U.S. Pat. No. 5,489,508 or U.S. Pat. No. 5,645,986. Inhibition of telomerase has been described as a specific means for the therapeutic control of tumor cells. Important efforts in modifying the activity of telomerase in association with cancerous diseases have been disclosed in EP 666 313, WO 97/37691, WO 99/50279, US 2002/0045588 A1 or WO 98/28442. However, the above general teachings do not disclose any concrete teachings as to technical activity to a person skilled in the art. While a substance or a molecule interacting with the entire sequence region encoding hTERT will result in a reduction of the corresponding telomerase activity, e.g. in a cell culture, such substances, however, are not suitable for application in organisms because they are normally too large in size, being attacked and destroyed by the immune system of the respective organism. Moreover, a large number of undesirable interactions or side effects may occur. The object of the invention was therefore to provide alternative, compact molecules that would undergo facile and effectively inhibiting interaction with selected specific structural units encoding telomerase.

The invention solves the above technical problem by providing a polynucleotide directed towards an mRNA of the catalytic subunit of human telomerase (hTERT), said polynucleotide undergoing specific interaction particularly with primary structures of said hTERT-mRNA in two target sequence regions, 2176 to 2250 and 2296 to 2393, according to the gene data base entry AF 015950. The numbers represent the corresponding nucleotide positions within the hTERT-mRNA (overall length: 4015 nucleotides), and this also applies to the following sections. Hence, the invention relates to the unexpected teaching that tumor-associated abnormal hTERT-mRNA expression patterns and telomerase activity levels can be counteracted by possible hTERT inhibition using the polynucleotides according to the invention. Said polynucleotides are directed towards well-defined hTERT-mRNA sequence motifs in a range of from 2000 to 2500. They may represent biological and/or chemical structures capable of interacting with the target sequence region in such a way that specific recognition/binding and interaction can be determined. More specifically, examples of polynucleotides can be nucleic acid constructs and derivatives thereof. Of course, it is also possible to use other recognition molecules such as antibodies, lectins, Affilines, aptamers, chelators and others instead of or in combination with said polynucleotides.

In a particularly preferred embodiment the polynucleotide specifically interacts with two target sequence regions, 2176 to 2250 and 2296 to 2393. Advantageously, particularly efficient inhibition of hTERT is possible in these sequence regions. Likewise preferred are shorter regions having changes within said target sequences or changed peripheral regions or various derivatizations/modifications/fusions /complex formations which may also be combined and/or coupled with other recognition molecules such as polynucleotides.

Owing to these preferred target sequence regions, it is possible for a person skilled in the art to provide, in particular, very small and/or compact polynucleotides which essentially do not interact with or are not affected by other structures, particularly structures of immunologic defense, within the cell tissue or the organism, but instead are capable of specifically interacting with the target sequence region of the hTERT-mRNA.

It is envisaged in another preferred embodiment of the invention that the sequence region or the recognition molecule, in particular the polynucleotide, be modified by addition, amplification, inversion, missense mutation, nonsense mutation, point mutation, deletion and/or substitution. In the polynucleotide, for example, the above modifications may result in binding thereof to the mRNA of the catalytic hTERT subunit with higher avidity or specificity. Obviously, however, it may also be envisaged that the polynucleotide binds with lower specificity or avidity. In the meaning of the invention, the mutations in the hTERT sequence region can be heritable or non-heritable changes, for example. The modifications can be such in nature that detection thereof is possible directly on the mRNA level or on the DNA level. For example, the mutations may also include mutations in connection with cytologically visible genome and/or chromosome mutations associated with changes of the hTERT. Such mutations may originate in such a way that portions of the chromosome are lost, doubled, present in reversed orientation, or transferred on other chromosomes. Of course, such a mutation may involve only one or a few adjacent base pairs, as is the case in a point mutation, for example. If, for example, a base pair is lost in the form of a deletion, or if a base pair is interposed in addition, as in insertion, the reading frame of the respective gene will be shifted to form a reading frame mutation. For example, in a substitution mutation in the meaning of the invention, one base is replaced by another, in which case the resulting consequences may be different:
(a) one codon may be converted into a synonymous codon, for example; or
(b) the mutation changes the codon specificity, resulting in incorporation of other amino acids; or
(c) the mutation causes termination of translation in a particular position, in which case the hTERT fragments being formed can be either inactive or active.

In another preferred embodiment the polynucleotide is a nucleic acid construct. Nucleic acid constructs in the meaning of the invention can be all structures based essentially on nucleic acids, or wherein the active center is based essentially on nucleic acids. The polynucleotide can be a component of complexes or formulations consisting of lipids, carbohydrates or proteins or peptides, e.g. in the form of a nanocapsule. The complex or formulation comprises a portion containing nucleic acids capable of interacting with hTERT. Various ways of providing such constructs are well-known to those skilled in the art.

In a preferred embodiment of the invention the nucleic acid construct is an antisense (AS) oligonucleotide (ON), a DNAzyme, a ribozyme, an siRNA and/or a peptide nucleic acid (PNA).

AS constructs are synthetically produced molecules allowing selective inhibition of the biosynthesis of selected proteins. For example, ONs, PNAs, ribozymes, DNAzymes are employed. The AS effect is based on sequence-specific hybridization of the constructs through Watson-Crick base pairing with the target mRNA encoding the protein to be repressed, resulting in prevention of protein synthesis via various mechanisms (Tab. 1).

TABLE 1
AS effects and their mechanisms of action

| Effect | Mechanism | | References |
|---|---|---|---|
| Inhibition of transcription | Binding of the AS constructs to genomic | | [Moser et |
| | DNA by Hoogsten triplex formation | | al.] |
| Modulation of RNA | a) | blocking of splicing sites results in prevention | [Kole et al., |
| processing | | of the splicing process | Crooke] |
| | b) | prevention of polyadenylation destabilizes | |
| | | the mRNA | |
| | c) | obstruction of mRNA transport into the | |
| | | cytoplasm | |
| Inhibition of translation | Competitive binding of the AS construct to | | [Boiziau et |
| | the target mRNA prevents initiation or elongation | | al.] |
| | process | | |
| Cleavage of target | a) | selective degradation of the RNA strand | [Crooke, |
| mRNA | | in RNA-DNA hybrids by RNase H endonuclease | Agrawal et |
| | b) | degradation of ss-RNA by RNase L endonuclease | al., Sun et |
| | | after activation by 2′,5′- | al.] |
| | | tetraadenylate-modified ON | |
| | c) | ribozyme/DNAzyme-catalyzed, sequence- | |
| | | specific cleavage of target | |
| | | mRNA | |

ss: single-stranded

Apart from other fields of application, the development of AS-ONs as therapeutic substances also represents a new promising therapeutical concept for oncologic diseases [Tamm et al.]. While conventional chemotherapy results in non-specific inhibition of cell proliferation, the AS therapy very specifically inactivates those mRNAs which represent the molecular basis or an essential component of degenerate, deregulated growth and tumor progression and may be responsible for the inhibition of the endogenous immune defense.

AS-ONs differ from other therapeutic agents, such as antibodies, toxins or immunotoxins, in that they are relatively small molecules with a molecular weight of normally about 5 kDa. The small size of the AS-ONs allows good tissue penetration. Furthermore, tumor blood vessels, as opposed to blood vessels of normal tissues, are known to be permeable to substances ranging in size between 4 to 10 kDa. Consequently, therapeutic AS-ONs are capable of penetrating tumor blood vessels in a well-directed fashion. Another advantage of these substances, e.g. with respect to antibodies almost exclusively effective against extracellular proteins, is that all proteins, in principle, i.e. cytoplasmic and membranous, as well as proteins located in the nucleus, can be attacked via the respective target mRNA.

At present, phospothioate AS-ONs relatively resistant to nuclease attack are being evaluated with respect to their potential as anti-cancer therapeutic agents in a number of clinical studies (phase I-III), with target mRNA molecules overexpressed in tumors being attacked.

When using phospothioate ONs (PS-ON), a number of unexpected, so-called “non-AS” effects have been observed which, in addition, may give rise to non-specific inhibition of cell growth. These effects strongly depend on the ON sequence or on specific sequence motifs, occurring due to strong polyanionic charge of the PS-ONs, which may result in binding of the PS-ONs to vital proteins. The negative effects mentioned above can be overcome particularly by using partially phosphothioate-modified AS-ONs, or by means of additional modifications, e.g. incorporation of ribonucleotides instead of deoxyribonucleotides. Partial terminal modification of ON constructs (preferably 2 to 5 bonds of the 3′ and 5′ nucleic acid terminus being modified) offers increased stability in the extra- and intracellular media of target cells (protection from degradation by exonucleases), especially in in vivo application. One positive side effect observed when using PS-ONs is their immunostimulatory effect which can be quite useful in supporting possible therapeutic success in some tumor applications.

To increase the stability and specificity of AS-ONs and reduce the “non-AS” effects, further chemical modifications can be employed, e.g. incorporation of 2′-O-methylribonucleotides, methylphosphonate segments, locked nucleic acids (methylene bridge between 2′ oxygen and 4′ carbon of ribose), replacement of cytosine by 5′-methylcytosine and/or 2′,5′-tetraadenylate modification.

Concerned in this context are partially modified ON constructs or those completely changed via the above chemical modifications.

Being catalytically active RNA molecules, ribozymes are capable of recognizing cellular RNA structures as substrates, cleaving them at a phosphodiester bond in a sequence-specific fashion. Recognition proceeds via AS branches which, owing to complementary sequences, allow hybridization with the target mRNA. Compared to AS-ONs, ribozymes have the fundamental advantage that a ribozyme molecule, being a true catalyst, is capable of reacting a large number of identical substrate molecules. Consequently, ribozymes are effective at substantially lower concentrations compared to ONs and, in addition,

lead to irreversible RNA degradation as a result of substrate cleavage [Sun et al.]. Amongst the types of ribozymes known to date, the hammerhead ribozyme (Review: Birikh et al., 1997; Tanner, 1999) is particularly interesting for such uses because it has catalytic activity even as a comparatively small molecule (about 30-50 nucleotides). For example, a highly effective trans-cleaving hammerhead ribozyme consists of no more than 14 conserved nucleotides in the catalytic domain and two variable ancestral sequences (each advantageously made up of 6 to 8 nucleotides) which, via Watson-Crick base pairing (in analogy to AS-ON), accomplish sequence-specific recognition of the substrate to be cleaved, subsequently inactivating the latter by cleavage of a phosphodiester bond. In this fashion, it is possible to construct a specifically cleaving hammerhead ribozyme for virtually any RNA molecule having a potential cleavage site with the minimum sequence requirement -NUX- and thus inhibit e.g. cellular mRNA or viral RNA. Other catalytic nucleic acids of the DNA type (e.g. DNAzymes) can be used in an analogous manner.

RNAi (RNA interference) is a new methodology allowing specific gene inhibition of target molecules on an mRNA level. To this end, double-stranded RNA molecules (small interference RNA, siRNA) with their 3′ overhangs two nucleotides in length, preferably consisting of thymidine nucleotides, must be transfected into cells. Initially, the siRNA constructs are associated with specific cellular proteins, followed by recognition of the target mRNA sequence on the basis of the complementarity of the AS-siRNA strand. The intrinsic endonuclease activity of the ribonucleoprotein complex allows specific degradation of the mRNA to be inhibited.

In one distinctive embodiment of the invention the AS-ON is a PS-ON or a nucleic acid construct modified with further chemical changes.

In another preferred embodiment of the invention the sequence region of the hTERT-mRNA, to which the polynucleotide is complementary, is selected from the group comprising 2183-2205, 2206-2225, 2315-2334, 2317-2336, 2324-2346, 2331-2350 and/or 2333-2352.

Advantageously, these sequence regions permit inhibition of hTERT expression. Inter alia, said inhibition allows suppression of diseases associated with the expression of this gene, such as tumors.

In another preferred embodiment of the invention the polynucleotide is immobilized. In the meaning of the invention, immobilization is understood to involve various methods and techniques to fix the polynucleotides on specific carriers. For example, immobilization can serve to stabilize the polynucleotides so that their activity would not be reduced or adversely modified by biological, chemical or physical exposure, especially during storage or in single-batch use. Immobilization of the polynucleotides allows repeated use under technical or clinical routine conditions; furthermore, the sample can be reacted with the polynucleotides in a continuous fashion. In particular, this can be achieved by means of various immobilization techniques, with binding of the polynucleotides to other polynucleotides or molecules or to a carrier proceeding in such a way that the three-dimensional structure in the active center of the corresponding molecules, especially of said polynucleotides, would not be changed. Advantageously, there is no loss in specificity to hTERT and in specificity of the actual binding reaction as a result of such immobilization. In the meaning of the invention, three basic methods can be used for immobilization:
(i) Crosslinking: in crosslinking, the polynucleotides are fixed to one another without adversely affecting their activity. Advantageously, they are no longer soluble as a result of crosslinking.
(ii) Binding to a carrier: binding to a carrier proceeds via adsorption, ionic binding or covalent binding, for example. Such binding may also take place inside microbial cells or liposomes or other membranous, closed or open structures. Advantageously, the polynucleotide is not adversely affected by such fixing. For example, carrier-bound multiple or continuous use thereof is possible with advantage in clinics in diagnosis or therapy.
(iii) Inclusion: inclusion in the meaning of the invention especially is inclusion in a semipermeable membrane in the form of gels, fibrils or fibers. Advantageously, encapsulated polynucleotides are separated from the surrounding sample solution by a semipermeable membrane in such a way that interaction with the catalytic subunit of human telomerase or with fragments thereof still is possible.

Various methods are available for immobilization, such as adsorption on an inert or electrically charged inorganic or organic carrier. For example, such carriers can be porous gels, aluminum oxide, bentonite, agarose, starch, nylon or polyacrylamide. Immobilization proceeds via physical binding forces, frequently involving hydrophobic interactions and ionic binding. Advantageously, such methods are easy to handle, having little influence on the conformation of the polynucleotides. Advantageously, binding can be improved as a result of electrostatic binding forces between the charged groups of the polynucleotides and the carrier, e.g. by using ion exchangers such as Sephadex. Another method is covalent binding to carrier materials. In addition, the carriers may have reactive groups forming homopolar bonds with amino acid side chains. Suitable groups in polynucleotides are carboxy, hydroxy and sulfide groups and especially the terminal amino groups of lysines. Aromatic groups offer the possibility of diazo coupling. The surface of microscopic porous glass particles can be activated by treatment with silanes and subsequently coated with polynucleotides. For example, hydroxy groups of natural polymers can be activated with bromocyanogen and subsequently coupled with polynucleotides. Advantageously, a large number of polynucleotides can undergo direct covalent binding with polyacrylamide resins. Inclusion in three-dimensional networks involves inclusion of the polynucleotides in ionotropic gels or other structures well-known to those skilled in the art. More specifically, the pores of the matrix are such in nature that the polynucleotides are retained, allowing interaction with the target molecules. In crosslinking, the polynucleotides are converted into polymer aggregates by crosslinking with bifunctional agents. Such structures are gelatinous, easily deformable and, in particular, suitable for use in various reactors. By adding other inactive components such as gelatin in crosslinking, advantageous improvement of mechanical and enzymatic properties is possible. In microencapsulation, the reaction volume of the polynucleotides is restricted by means of membranes. For example, microencapsulation can be carried out in the form of an interfacial polymerization. Owing to the immobilization during microencapsulation, the polynucleotides are made insoluble and thus reusable. In the meaning of the invention, immobilized recognition molecules, especially polynucleotides, are all those recognition molecules or polynucleotides being in a condition that allows reuse thereof. Restricting the mobility and solubility of the polynucleotides by chemical, biological or physical means advantageously results in lower process cost.

The invention also relates to a pharmaceutical composition comprising the polynucleotides of the invention, optionally in combination with a pharmaceutically tolerable carrier. More specifically, the pharmaceutical carrier may comprise additional materials and substances such as medical and/or pharmaceutical-technical adjuvants. For example, medical adjuvants are materials used as ingredients in the production of pharmaceutical compositions. Pharmaceutical-technical adjuvants serve to suitably formulate the drug or pharmaceutical composition and, if required during the production process only, can even be removed thereafter, or they can be part of the pharmaceutical composition as pharmaceutically tolerable carriers. Formulation of the pharmaceutical composition is optionally effected in combination with a pharmaceutically tolerable diluent. For example, the diluents can be phosphate-buffered saline, water, emulsions such as oil/water emulsions, various types of detergents, sterile solutions, and the like. The pharmaceutical composition can be administered in association with a gene therapy, for example.

In the meaning of the invention, gene therapy is a form of treatment using natural or recombinantly engineered nucleic acid constructs, single gene sequences or complete gene or chromosome sections or encoded transcript regions, derivatives/modifications thereof, with the objective of a biologically based and selective inhibition or reversion of disease symptoms and/or the causal origin thereof, in special cases this being understood to involve inhibition of a target molecule on a nucleic acid level, especially transcript level, which has been overexpressed in the course of a disease.

For example, gene therapy may also be effected using suitable vectors such as viral vectors or/and complex formation with lipids or dendrimers. In particular, gene therapy may also proceed via packaging in protein coats. Furthermore, the polynucleotide can be fused or complexed with another molecule supporting the directed transport to the target site, uptake in and/or distribution inside a target cell. The kind of dosage and route of administration can be determined by the attending physician according to clinical requirements. As is familiar to those skilled in the art, the kind of dosage will depend on various factors, such as size, body surface, age, sex, or general and pathognomonic health condition of the patient, but also on the particular agent being administered, the time period and type of administration, and on other medications possibly administered in parallel, especially in a combination therapy.

The invention also relates to a kit comprising the polynucleotide and/or the pharmaceutical composition. Furthermore, the invention also relates to an array comprising the polynucleotide and/or the pharmaceutical composition. Kit and array can be used in the diagnosis and/or therapy of diseases associated with the function of the catalytic subunit of human telomerase. The invention also relates to the use of said polynucleotide, said kit, said array in the diagnosis, prophylaxis, reduction, therapy, follow-up and/or aftercare of diseases associated with cell growth, differentiation and/or division.

In a preferred embodiment the disease associated with cell growth, differentiation and/or division is a tumor. In a particularly preferred fashion the tumor is a solid tumor and/or blood or lymphatic node cancer.

More specifically, the tumors in the meaning of the invention, which can be of epithelial or mesodermal origin, can be benign or malignant types of tumors in organs such as lungs, prostate, urinary bladder, kidneys, esophagus, stomach, pancreas, brain, ovaries, skeletal system, with adenocarcinoma of breast, prostate, lungs and intestine, bone marrow cancer, melanoma, hepatoma, ear-nose-throat tumors in particular being explicitly preferred as members of so-called malignant tumors. In the meaning of the invention, the group of blood or lymphatic node cancer types includes all forms of leukemias (e.g. in connection with B cell leukemia, mixed-cell leukemia, null cell leukemia, T cell leukemia, chronic T cell leukemia, HTLV-II-associated leukemia, acute lymphocytic leukemia, chronic lymphocytic leukemia, mast cell leukemia, and myeloid leukemia) and lymphomas. Examples of mesenchymal malignant tumors (so-called bone and soft-tissue sarcomas) are: fibrosarcoma; malignant histiocytoma; liposarcoma; hemangiosarcoma; chondrosarcoma and osteosarcoma; Ewing sarcoma; leio- and rhabdomyosarcoma, synovialsarcoma; carcinosarcoma. As further types of tumors which, in the meaning of the invention, will be summarized under the term of “neoplasms” the following are preferred: bone neoplasms, breast neoplasms, neoplasms of the digestive system, colorectal neoplasms, liver neoplasms, pancreas neoplasms, hypophysis neoplasms, testicle neoplasms, orbital neoplasms, neoplasms of head and throat, of the central nervous system, neoplasms of the hearing organ, pelvis, respiratory tract and urogenital tract.

In another preferred embodiment the cancerous disease or tumor being treated or prevented is selected from the group of: tumors of the ear-nose-throat region, comprising tumors of the inner nose, nasal sinus, nasopharynx, lips, oral cavity, oropharynx, larynx, hypopharynx, ear, salivary glands, and paragangliomas, tumors of the lungs, comprising non-parvicellular bronchial carcinomas, parvicellular bronchial carcinomas, tumors of the mediastinum, tumors of the gastrointestinal tract, comprising tumors of the esophagus, stomach, pancreas, liver, gallbladder and biliary tract, small intestine, colon and rectal carcinomas and anal carcinomas, urogenital tumors comprising tumors of the kidneys, ureter, bladder, prostate gland, urethra, penis and testicles, gynecological tumors comprising tumors of the cervix, vagina, vulva, uterine cancer, malignant trophoblast disease, ovarian carcinoma, tumors of the uterine tube (Tuba Faloppii), tumors of the abdominal cavity, mammary carcinomas, tumors of the endocrine organs, comprising tumors of the thyroid, parathyroid, adrenal cortex, endocrine pancreas tumors, carcinoid tumors and carcinoid syndrome, multiple endocrine neoplasias, bone and soft-tissue sarcomas, mesotheliomas, skin tumors, melanomas comprising cutaneous and intraocular melanomas, tumors of the central nervous system, tumors during infancy, comprising retinoblastoma, Wilms tumor, neurofibromatosis, neuroblastoma, Ewing sarcoma tumor family, rhabdomyosarcoma, lymphomas comprising non-Hodgkin lymphomas, cutaneous T cell lymphomas, primary lymphomas of the central nervous system, Hodgkin's disease, leukemias comprising acute leukemias, chronic myeloid and lymphatic leukemias, plasma cell neoplasms, myelodysplasia syndromes, paraneoplastic syndromes, metastases with unknown primary tumor (CUP syndrome), peritoneal carcinomatosis, immunosuppression-related malignancy comprising AIDS-related malignancies such as Kaposi sarcoma, AIDS-associated lymphomas, AIDS-associated lymphomas of the central nervous system, AIDS-associated Hodgkin disease, and AIDS-associated anogenital tumors, transplantation-related malignancy, metastasized tumors comprising brain metastases, lung metastases, liver metastases, bone metastases, pleural and pericardial metastases, and malignant ascites.

In a distinctive embodiment of the invention the solid tumor is a tumor of the urogenital tract and/or gastrointestinal tract.

In another particularly preferred embodiment of the invention it is envisaged that the tumor is a colon carcinoma, stomach carcinoma, pancreas carcinoma, a colon cancer, small intestine cancer, an ovarian carcinoma, cervical carcinoma, a lung cancer, a renal cell carcinoma, a brain tumor, a head- throat tumor, a liver carcinoma and/or a metastase of the above tumors/carcinomas.

In another particularly preferred embodiment the solid tumor is a mammary, bronchial, colorectal and/or prostate carcinoma.

In a most preferred embodiment the tumor of the urogenital tract is a bladder carcinoma (BCa). In the Federal Republic of Germany, BCa represents the fourth most frequent form of cancer and the seventh most frequent cause of cancer death in males. TUR-B as a general primary therapy of BCa allows organ-preserving removal of superficial tumors. Despite such histopathologically defined complete removal of the tumor, a relatively high percentage of patients, being from 50 to 70%, experience a relapse within two years [Stein et al.]. One problem in diagnosis and therapy is the synchronous or metachronous multifocal appearance of tumor centers, which may be a possible cause of the appearance of relapses remote from the resected primary tumor location [Sidransky et al.]. In cases of appearing relapses or tumors primarily classified as superficial, the TUR-B is normally followed by a long-term prophylaxis using an immunotherapeutic (bacillus Calmette Guerin; BCG) or chemotherapeutic agent (e.g. mitomycin C, taxol, gemcitabin/cisplatin). Patients with muscle-invasive BCa and dedifferentiated superficial tumors, who experience relapse despite such therapy, are normally treated with radical cystectomy or, preserving the bladder, by means of mono-/polychemo-, immuno- or radiotherapy or combined procedures of these methods. Due to their relatively unspecific mechanisms of action, chemical, immune or radiation treatments are accompanied by high therapy-induced toxicity.

Due to the importance of BCa in health policy (especially in Western industrial nations), lack of tumor-specific markers, and well-known tumor-biological and cellular heterogeneity of the tumor, there is an intense search in the field of clinical research on BCa, particularly with the aim of identifying new or/and supplementing therapeutical options.

In a distinctive embodiment of the invention the polynucleotide, the pharmaceutical composition, the kit and/or the array are used in a follow-up essentially representing monitoring the effectiveness of an anti-tumor treatment. Furthermore, it is preferred that the polynucleotide be used in a combination therapy, especially for the treatment of tumors. In a particularly preferred fashion, said combination therapy comprises a chemotherapy, a treatment with cytostatic agents and/or a radiotherapy. In a particularly preferred embodiment of the invention the combination therapy is an adjuvant, biologically specific form of therapy, and in a particularly preferred fashion, said form of therapy is an immune therapy. Furthermore, in a particularly preferred fashion the combination therapy comprises a gene therapy and/or a therapy using a polynucleotide against the same or other target molecule. Various combination therapies, especially for the treatment of tumors, are well-known to those skilled in the art. For example, a treatment with cytostatic agents or e.g. irradiation of a particular tumor area can be envisaged within the scope of a combination therapy, and this treatment is combined with a gene therapy, using the polynucleotide of the invention as an anticancer agent. However, the polynucleotide according to the invention can also be used in combination with other polynucleotides directed against the same or other target molecule. Accordingly, the use of the polynucleotide for increasing the sensitivity of tumor cells to cytostatic agents and/or radiation can be particularly preferred. Furthermore, a preferred use of the polynucleotide is in inhibiting the vitality, the proliferation rate of cells and/or inducing apoptosis and cell cycle arrest.

Without intending to be limiting, the invention will be explained in more detail with reference to the examples.

### EXAMPLE 1

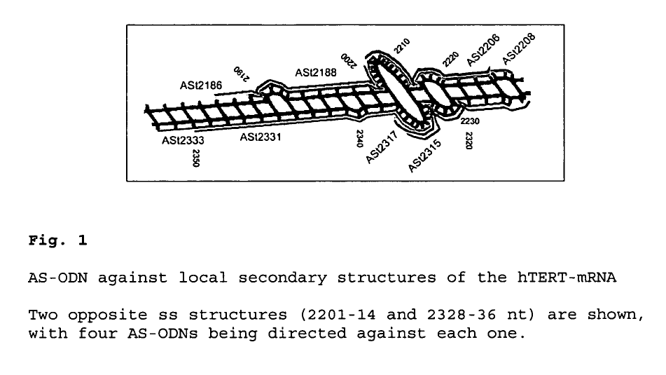

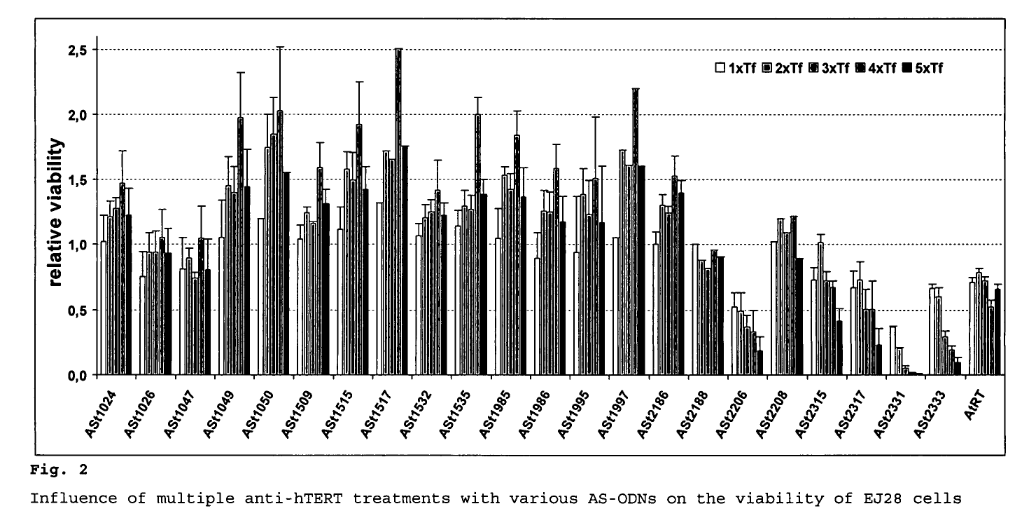

Following transfection, especially when using five specific anti-hTERT-AS constructs (cf. Table 2), the easily transfectable human bladder carcinoma cell line EJ28 showed immediate and continuous reduction of its viability by more than 65% compared to the nonsense (NS) control (FIG. 2). It was remarkable to observe that four of the most effective constructs were directed against one single mRNA sequence motif.

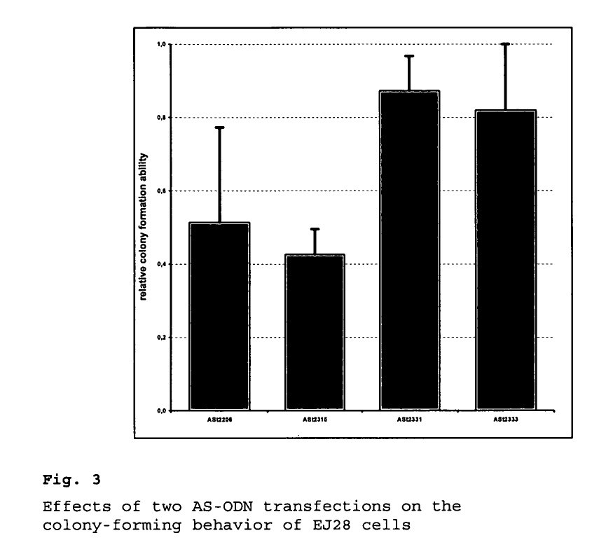

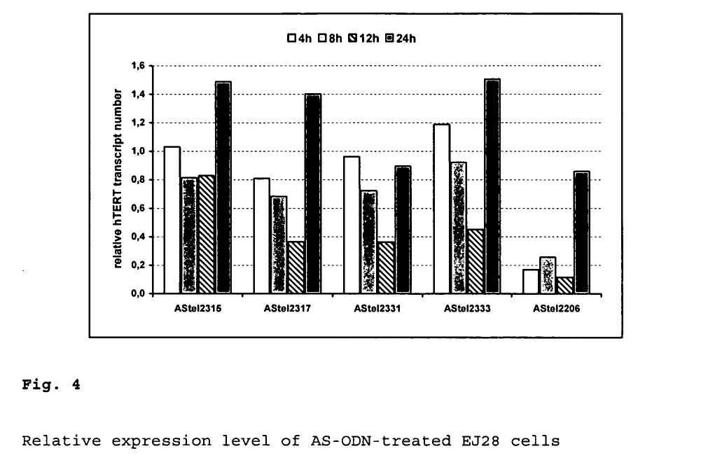

After four of five treatments with the construct AStel2331-50, virtually no living cells could be detected in the culture vessel anymore. In contrast, treatment of telomerase-negative human fibroblasts gave no significant differences between AS- and NS-ON-treated cells, thus indirectly proving specificity of the AS-ON effect on the BCa cell line EJ28 (data not shown). AS-specific efficacy was subsequently investigated in detail: in accordance with the viability test, an inhibiting effect of the five AS-ONs with respect to proliferation and cell colony-forming behavior could be demonstrated (FIG. 3). Moreover, AS-specific reduction of the cell percentage in the DNA synthesis phase (up to about 30%) towards G1 arrest could be detected (data not shown). Evidence of the AS-specific effect of the AS-ONs directed against the target motifs was furnished in the form of a significant and time-dependent reduction of the hTERT transcript level (FIG. 4). Correspondingly, there was also a repression of the hTERT protein expression. Furthermore, as a result thereof, the telomerase activity of the EJ28 cells was inhibited by more than 60% (data not shown).

In addition, the AS-ODN-specific effects with respect to inhibition of growth and proliferation were demonstrated on other human BCa cell lines (Kraemer et al.).

### EXAMPLE 2

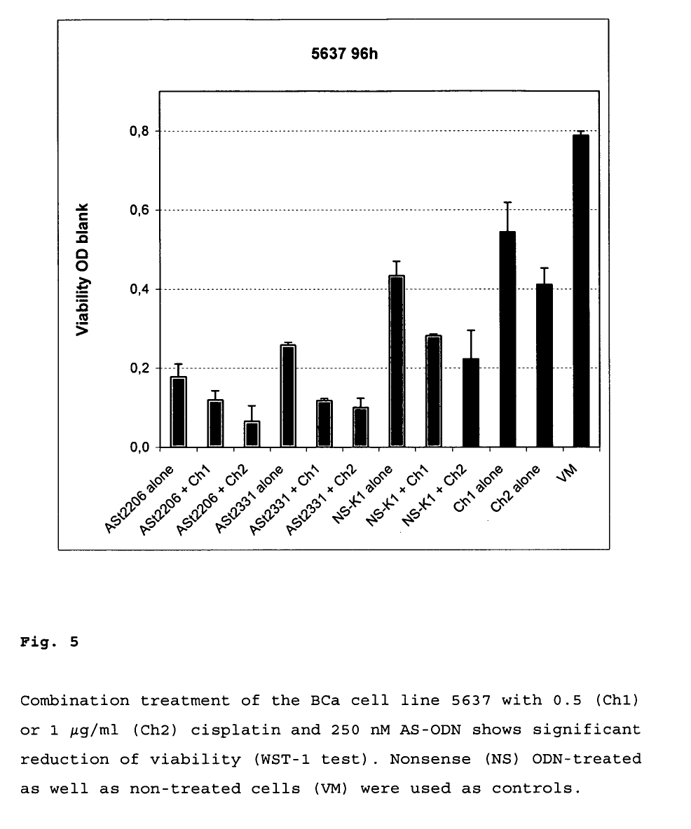

Unexpectedly, a significant enhancing effect caused by addition of single polynucleotides in the meaning of the invention was observed in experiments on the effect of various chemotherapeutic agents (mitomycin C, cisplatin, gemcitabin) on the growth behavior of different BCa cell lines (data not shown). Using the example of the easily transfectable BCa cell line 5637, it was possible to detect a significant increase of the viability-inhibiting effect of the cisplatin chemotherapeutic agent at two different dosages with the AS-ON constructs AStel2206 and AStel2331 (FIG. 5).

TABLE 2

hTERT-AS and NS-ON: nucleotide and target
sequences

| | | |
|---|---|---|
| Designation¹ | ss motif² | Sequence³ (5′→3′) |

| | | |
|---|---|---|
| AS-ON | | |
| ASte12206-2225 | 2191-2224 | tgtcctgggggatggtgtcg |
| ASte12315-2334 | 2318-2346 | ttgaaggccttgcggacgtg |
| ASte12317-2336 | | tcttgaaggccttgcggacg |
| ASte12331-2350 | | ggtagagacgtggctcttga |
| ASte12333-2352 | | aaggtagagacgtggctctt |
| NS-ON | | |
| NS-K2 | — | cagtctcagtactgaagctg |
| NS-K3 | | cagcttcagtactgagactg |

¹The designation includes the sequence region of the hTERT-mRNA (Acc. No.: AF015950) to which the respective AS-ON is complementary.
²The illustrated motifs include 10 nt double-stranded RNA at each 5′ and 3′ terminus.
³The nucleotides in bold type represent the AS-ON region which is complementary to the actual ss region of the target motif.

### REFERENCES

Agrawal S, Zhao Q: Antisense therapeutics. Curr Opin Chem Biol (1998) 2: 519-28.
Beattie T L, Zhou W, Robinson M O, Harrington L: Reconstitution of human telomerase activity in vitro. Curr Biol (1998) 8: 177-80.
Boiziau C, Kurfurst R, Cazenave C, Roig V, Thuong N T, Toulme J J: Inhibition of translation initiation by antisense oligonucleotides via an RNase-H independent mechanism. Nucleic Acids Res (1991) 19: 1113-9.
Crooke S T: Molecular mechanisms of action of antisense drugs. Biochim Biophys Acta (1999) 1489: 3144.
Greider C W, Blackburn E H: Identification of a specific telomere terminal transferase activity in Tetrahymena extracts. Cell (1985) 43: 405-13.
Harley C B: Telomere loss: mitotic clock or genetic time bomb? Mutat Res (1991) 256: 271-82.
Ito H, Kyo S, Kanaya T, Takakura M, Inoue M, Namiki M: Expression of human telomerase subunits and correlation with telomerase activity in urothelial cancer. Clin Cancer Res (1998) 4: 1603-8.
Kim N W, Piatyszek M A, Prowse K R, Harley C B, West M D, Ho P L, Coviello G M, Wright W E, Weinrich S L, Shay J W: Specific association of human telomerase activity with immortal cells and cancer. Science (1994) 266: 2011-5.
De Kok J B, Schalken J A, Aalders T W, Ruers T J, Willems H L, Swinkels D W: Quantitative measurement of telomerase reverse transcriptase (hTERT) mRNA in urothelial cell carcinomas. Int J Cancer (2000) 87: 217-20.
Kole R, Sazani P: Antisense effects in the cell nucleus: modification of splicing. Curr Opin Mol Ther (2001) 3: 229-34.
Kraemer K, Fuessel S, Schmidt U, Kotzsch M, Schwenzer B, Wirth M P, Meye A: Antisense-mediated hTERT Inhibition Specifically Reduces the Growth of Human Bladder Cancer Cells. Clin Cancer Res (2003) 9: 3794-800.
Levy M Z, Allsopp R C, Futcher A B, Greider C W, Harley C B: Telomere end-replication problem and cell aging. J Mol Biol (1992) 225: 951-60.
Moser H E, Dervan P B: Sequence-specific cleavage of double helical DNA by triple helix formation. Science (1987) 238: 645-50.
Stein J P, Grossfeld G D, Ginsberg D A, Esrig D, Freeman J A, Figueroa A J, Skinner D G, Cote R J: Prognostic markers in bladder cancer: a contemporary review of the literature. J Urol (1998) 160: 645-59.
Sun L Q, Cairns M J, Saravolac E G, Baker A, Gerlach W L: Catalytic nucleic acids: from lab to applications. Pharmacol Rev (2000) 52: 32547.
Tamm I, Dorken B, Hartmann G: Antisense therapy in oncology: new hope for an old idea? Lancet (2001) 358: 489-97.

1. A polynucleotide directed towards a gene of a catalytic subunit of human telomerase, characterized in that
the polynucleotide specifically interacts with the mRNA of the catalytic subunit of human telomerase in at least two target sequence regions, 2176 to 2250 and 2296 to 2393, in accordance with accession number AF015950.

2. The polynucleotide according to claim 1, wherein
the polynucleotide interacts with target sequence regions selected from the group comprising 2183-2205, 2206-2225, 2315-2334, 2317-2336, 2324-2346, 2331-2350 and/or 2333-2352.

3. The polynucleotide according to claims 1, wherein
the sequence region and/or the polynucleotide is modified by addition, amplification, inversion, missense mutation, nonsense mutation, point mutation, deletion and/or substitution.

4. The polynucleotide according to claims 1, wherein
the polynucleotide is immobilized.

5. The polynucleotide according to claims 1, wherein
the polynucleotide is a nucleic acid construct or a derivative thereof.

6. The polynucleotide according to claim 5, wherein
the polynucleotide is fused or complexed with another molecule supporting the directed transport to the target site, the uptake in and/or distribution inside a target cell.

7. The polynucleotide according to claim 5, wherein
the nucleic acid construct is an antisense oligonucleotide, a DNAzyme, a peptide nucleic acid, a ribozyme and/or an siRNA.

8. The polynucleotide according to claim 7, wherein
the antisense oligonucleotide is modified by phosphothioate bonds and/or other chemical modifications.

9. The polynucleotide according to claims 1, wherein
the sequence region of the hTERT-mRNA, to which the polynucleotide is complementary, is selected from the group comprising 2183-2205, 2206-2225, 2315-2334, 2317-2336, 2324-2346, 2331-2350 and/or 2333-2352.

10. A pharmaceutical composition comprising a polynucleotide according to claims 1 in combination with a pharmaceutically tolerable carrier.

11. A kit comprising
a polynucleotide according to claims 1 and a pharmaceutically tolerable carrier.

12. (canceled)

13. Method for diagnosis, prophylaxis, therapy, follow-up and/or aftercare of diseases associated with cell growth, differentiation and/or division, comprising using a polynucleotide according to claim 1, optionally in combination with a pharmaceutically tolerable carrier.

14. The method according to the preceding claim, wherein the disease is a tumor.

15. The use method according to claim 14, wherein
the tumor is a solid tumor or a leukemia.

16. The method according to claim 15, wherein
the solid tumor is a tumor of the urogenital tract and/or gastrointestinal tract.

17. (canceled)

18. (canceled)

19. The use method according to claim 16, wherein
the tumor of the urogenital tract is a bladder carcinoma and/or a metastase of said tumor.

20. The method according to claim 13, wherein
the follow-up is monitoring the effectiveness of an anti-tumor treatment.

21. The method according to claims 13, wherein
the polynucleotide is used in a combination therapy.

22. (canceled)

23. The method according to claim 22, wherein
the combination therapy is comprises an adjuvant biologically specified form of therapy.

24. The method according to claim 23, wherein
said form of therapy is an immune therapy.

25. The method according to claims 21, wherein
the combination therapy is a gene therapy and/or a therapy using a polynucleotide against the same or other target molecule.

26. The method according to claims 13 for increasing the sensitivity of tumor cells to cytostatic agents and/or radiation.

27. Method for inhibiting the vitality, the proliferation rate of cells, for inducing apoptosis and/or cell cycle arrest, comprising, the step of using a polynucleotide according to claim 1.
